(19) United States
(12) Patent Application Publication
(10) Pub. No.: US 2017/0348479 A1
(43) Pub. Date: Dec. 7, 2017

(54) ADHESIVE SYSTEM FOR DRUG DELIVERY DEVICE

(71) Applicants: Bryan Choate, Salem, MA (US); Amanda Callaghan, Hopkinton, MA (US); Bryan Dillon, Jefferson, MA (US); David Clare, Georgetown, MA (US)

(72) Inventors: Bryan Choate, Salem, MA (US); Amanda Callaghan, Hopkinton, MA (US); Bryan Dillon, Jefferson, MA (US); David Clare, Georgetown, MA (US)

(21) Appl. No.: 15/172,336

(22) Filed: Jun. 3, 2016

(51) Int. Cl. A61M 5/158

(52) U.S. Cl. CPC A61M 5/158; A61M 2005/1586

(57) ABSTRACT

Systems and methods for securing a drug delivery system to the body of a person are provided. The drug delivery system can include a pad coupled to a bottom of the drug delivery system. A bottom surface of the pad can include an adhesive for attaching to a user of the drug delivery system. An adhesive overlay can be positioned over the drug delivery system to improve attachment of the drug delivery system to the patient. The adhesive overlay can include an interior cutout that fits over the drug delivery system. A first portion of the adhesive overlay can adhere to the pad of the drug delivery system and a second portion of the adhesive overlay can adhere to the skin of the user. The adhesive overlay can have a larger surface area than the pad to improve the attachment of the drug delivery system to the user.

### TECHNICAL FIELD

Embodiments herein generally relate to medication delivery and, more particularly, to systems and methods for attaching wearable medication delivery devices to the body of a patient.

### BACKGROUND

A person may wear a medical device on his or her body for any of a variety of reasons, such as insulin injection, heart rate monitoring, or other reason. Many wearable medication delivery devices use an adhesive pad to adhere the wearable medication delivery device to the skin of the user. Over time, portions of the adhesive pad can become decoupled from the body of the user. Increased humidity or perspiration of the user can also weaken the ability of the adhesive pad from adhering to the user. Portions of the adhesive pad lifting up from the patient can be uncomfortable to the patient or can cause the wearable medication delivery device to fall off the user. A need therefore exists for an improved system and method for securing wearable medical devices to a user.

### BRIEF DESCRIPTION OF THE DRAWINGS

In the drawings, like reference characters generally refer to the same parts throughout the different views. In the following description, various embodiments of the present invention are described with reference to the following drawings, in which:

FIG. 1 illustrates a conventional drug delivery system.

FIG. 2 illustrates an adhesive overlay.

FIG. 3 illustrates application of the adhesive overlay of FIG. 2 onto the drug delivery system of FIG. 1.

FIG. 4 illustrates the adhesive overlay attached to the drug delivery system.

### DETAILED DESCRIPTION

Various embodiments include systems and methods for securing a wearable medical device to the body of a person. The wearable medical device can be a drug delivery system. The drug delivery system can be a disposable drug delivery system. The drug delivery system can include a pad coupled to a bottom of the drug delivery system. A bottom surface of the pad can include an adhesive for attaching to a user of the drug delivery system. An adhesive overlay can be positioned over the drug delivery system to improve attachment of the drug delivery system to the patient. The adhesive overlay can include an interior cutout that fits over the drug delivery system. A first portion of the adhesive overlay can adhere to the pad of the drug delivery system and a second portion of the adhesive overlay can adhere to the skin of the user. The adhesive overlay can have a larger surface area than the pad to improve the attachment of the drug delivery system to the user.

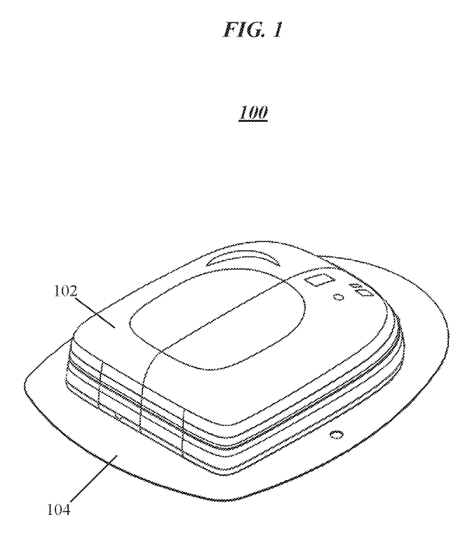

FIG. 1 illustrates a conventional medical system 100. The medical system 100 can be a wearable medical system 100 such as, for example, a medicine or drug delivery system 100. The drug delivery system 100 can include a drug delivery device 102 and a pad 104. The pad 104 can be coupled or attached to a bottom surface of the drug delivery device 102. The pad 104 can be an adhesive pad. Specifically, the pad 104 can include an adhesive positioned on a bottom surface of the pad 104. The adhesive of the pad 104 can be used to attach the drug delivery system 100 to a user. As an example, the pad 104 can be attached to the skin of a user. As shown in FIG. 1, the pad 104 can have a shape or profile that is larger than the drug delivery device 102. That is, an outer portion of the pad 104 can extend beyond an outer perimeter or profile of the drug delivery device 102.

The drug delivery device 102 can be any drug delivery device for providing any drug or medicine to a patient or user. In various embodiments, the drug delivery device 102 can be a disposable drug delivery device 102 intended for limited use. In various embodiments, the drug delivery device can be an insulin delivery device. The drug delivery device 102 can include additional components not shown in FIG. 1 for delivering one or more drugs or medicines to a user. In various embodiments, the drug delivery device 102 can be an OmniPod® (Insulet Corporation, Billerica, Mass.) insulin delivery device. The drug delivery device 102 can be a drug delivery device such as those described in U.S. Pat. No. 7,303,549, U.S. Pat. No. 7,137,964, or U.S. Pat. No. 6,740,059, each of which is incorporated herein by reference in its entirety. The drug delivery system 100 can be intended for use over a few days (e.g., approximately 3 days and/or approximately 80 hours). For example, the drug delivery system 100 can be positioned onto a user, used for a limited an amount of time, and then removed and replaced with another drug delivery system 100.

As discussed above, the adhesive of the pad 104 can be used to attach or couple the drug delivery system 100 to a user's skin. During the intended period of use of the drug delivery system 100, an outer rim or edge of the pad 104 can become decoupled from a user. That is, the outer edge of the pad 104 can begin to lift off from the user. Edge lift from the pad 104 can be due to a number of factors such as, for example, the adhesive of the pad 104 losing strength over time. Further, edge lift of the pad 104 can be caused or exacerbated by increases in humidity or by perspiration of a user (e.g., during warm summer months or periods of high activity of the user).

Reduction in the adhesive strength of the pad 104 and/or edge lift of the pad 104 can cause a drug delivery system 100 to fall off a user, can feel uncomfortable to a user, and or can otherwise be a nuisance.

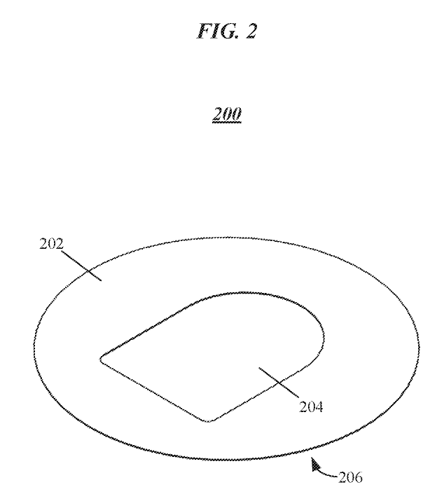

FIG. 2 illustrates an adhesive overlay 200. The adhesive overlay 200 can be used with the drug delivery system 100 to improve adhesion or coupling of the drug delivery system 100 to a user. The adhesive overlay 200 can include a layer of material 202. The layer of material 202 can have an outer shape. The outer shape can be any shape such as, for example, circular (as shown in FIG. 2) or elliptical. In various embodiments, the outer shape can have an amorphous shape, can be customizable, and/or can shaped like any of a variety of graphical depictions (e.g., as an animal, a football, or other shape). The adhesive overlay 200 can include a cutout 204 in the layer of material 202. The cutout 204 can be positioned towards an interior of the layer of material 202. The cutout 204 can therefore be considered to be an internal cutout of the additive overlay 200.

The cutout 204 can be of any shape and size. In various embodiments, the cutout 204 can have a shape or profile that is substantially identical or that substantially matches a shape or profile of the drug delivery device 102 (e.g., a profile or shape of the drug delivery device 102 when viewed from above). For example, the cutout 204 and the drug delivery device 102 can each have elliptical shapes. Further, the shape and sizing of the cutout 204 can be approximately the same as the drug delivery device 102. For example, the cutout 204 and the drug delivery 102 can each have elliptical shapes that are of substantially the same size. In various other embodiments, the size of the cutout 204 can be slightly larger than the size of the drug delivery device 102. Under either scenario, the cutout 204 can accordingly have a size and shape such that the cutout 204 can fit over the drug delivery device 102 (i.e., such that the drug delivery device 102 can fit through the cutout 204). The adhesive overlay 200 can be larger than the drug delivery system 100. Accordingly, when the adhesive overlay 200 is positioned over the drug delivery system 100 to fit the drug delivery device 102 through the cutout 204, an outer portion of the layer of material 102 can be positioned over the pad 104 and can also extend beyond the pad 104.

A bottom surface 206 of the layer of material 204 can include an adhesive. The adhesive on the bottom surface 206 of the layer of material 204 can attach to the pad 104 and can also attach the user. In various embodiments, a portion of the adhesive overlay 200 can be attached to the pad 104 and a portion of the adhesive overlay 200 can be attached to the user. As such, the adhesive overlay 200 can be used to further strengthen the coupling of the drug delivery system 100 to a user.

The layer of material 202 can be made of the same material as the pad 104 or can be made of a different material. In various embodiments, the layer of material 202 can be made of a polyester non-woven material. In various embodiments, the layer of material 202 can be made of a clear plastic film such as, for example, polyethylene. In various embodiments, the layer of material 202 can be flexible or can be stretchable.

The adhesive positioned on the bottom surface 206 of the layer of material 202 can be any adhesive including the same adhesive used on a bottom surface of the pad 104. In various embodiments, the adhesive can be an acrylic. In various embodiments, the adhesive positioned on the bottom surface 206 of the layer of material 202 can be stronger than the adhesive used on the bottom surface of the pad 104 (e.g., in terms of tensile stress required to remove the adhesive from a surface). In various embodiments, the adhesive positioned on the bottom surface 206 of the layer of material 202 can be as strong or can be weaker than the adhesive used on the bottom surface of the pad 104. Variable adhesive strengths of the adhesive on the bottom surface 206 of the layer of material 202 may be desired by different users depending on user applications (e.g., such as exercising, living in warmer climates, etc.).

The adhesive overlay 200 can decrease edge lift by the pad 104, can increase the adhesive strength of the pad 104, and/or can improve adhesion of the drug delivery system 100 to a user. The adhesive overlay 200 can be constructed by die cutting a piece of material or adhesive into a predetermined shape that can accommodate the drug delivery device 102. The cutout 204 can be formed to precisely fit over the drug delivery device 102 or can be slightly larger than the drug delivery device 102. If the cutout is formed to be slightly larger than the drug delivery device 102, then a portion of the pad 104 may remain exposed or uncovered when the adhesive overlay 200 is positioned over the drug delivery system 100. As shown in FIG. 2, since the adhesive overlay 200 is larger than the pad 104, adhesion to a user can be improved by the increased surface area provided by the adhesion overlay 200 and by coupling a portion of the adhesive overlay 200 to the pad 104 and a portion of the adhesive overlay 200 to a user.

The bottom surface 206 can be covered with a release liner prior to use. For example, a release liner can protect the adhesive applied to the bottom surface 206 until installation of the adhesive overlay 200. The layer of material 202 can have a thickness of less than 1 millimeter (mm). The top surface of the layer of material 202 can be of a variety of colors. In various embodiments, the top surface of the layer of material 202 can include any number of different colors, patterns, lettering, and/or numbering. For example, the top surface of the layer of material 202 can be customizable by a user to include various graphical and/or textual designs.

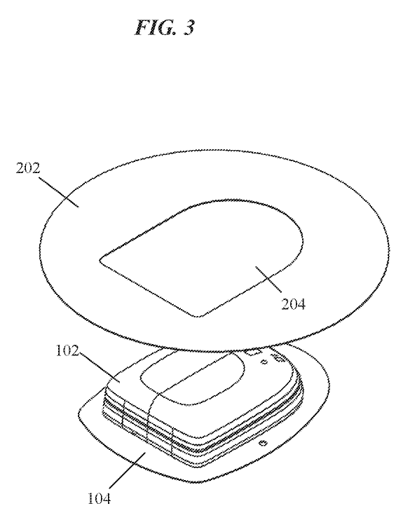

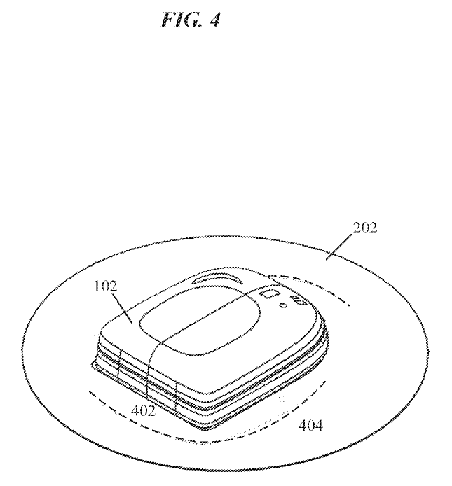

FIGS. 3 and 4 illustrate an application of the adhesive overlay 200. Specifically, FIG. 3 illustrates the adhesive overlay 200 just prior to being fit over the drug delivery system 100. Prior to application of the adhesive overlay 200, a release liner (not shown in FIG. 3) of the adhesive overlay 200 can be removed. The release liner can be attached or coupled to the bottom surface 206 of the adhesive overlay 200.

After removing the release liner, the adhesive overlay 200 can be positioned over the drug delivery system 100. Specifically, the interior cutout 204 can be positioned over the drug delivery device 102. Accordingly, as the adhesive overlay 200 is lowered further, the drug delivery device 102 can fit through the cutout 204.

As shown in FIG. 3, the shape or profile of the cutout 204 can be substantially the same as the shape or profile of the drug delivery device 102. The cutout 204 can be approximately the same size as the profile of the drug delivery device 102 or can be larger. In various embodiments, the cutout 204 is at least as large as the shape or profile of the drug delivery device 102 such that the drug delivery device 102 can fit through the cutout 104 with the cutout 104 abutting against or positioned next to a base of the drug delivery device (i.e., where the pad 104 and drug delivery device 102 are coupled or attached).

FIG. 4 illustrates the adhesive overlay 200 attached or adhered to the drug delivery system 100 and attached adhered to a user. For purposes of explanation, portions of an outline of the pad 104 are shown in phantom (i.e., in dashed lines). As shown in FIG. 4, the layer of material 202 is positioned over the pad 104. An interior or inner portion 402 of the layer of material 102 is positioned over the pad 104. The inner portion 402 can be coupled to or adhered to the pad 104. An exterior or outer portion 404 of the layer of material 202 can extend beyond the pad 104. The outer portion 404 can be adhered to or coupled to a user (e.g., the skin of the user).

As shown in FIG. 4, the layer of material 202 fits up against the base of the drug delivery device 102 since the cutout 204 can be shaped precisely to fit over the drug delivery device 102. In various embodiments, the cutout 204 can be larger than the drug delivery device 102 such that a gap between layer of material 202 and the drug delivery device 102 exposes a portion of the pad 104.

Certain embodiments were described above. It is, however, expressly noted that the disclosure is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

1. An apparatus, comprising:
a layer of material having an outer shape and an interior cutout; and
an adhesive positioned on a bottom surface of the layer of material, wherein the interior cutout has a shape substantially identical to a shape of a drug delivery device.

2. The apparatus of claim 1, wherein the drug delivery device fits through the interior cutout, wherein an inner portion of the bottom surface of the layer of material is positioned over an adhesive pad of the drug delivery device and an outer portion of the bottom surface of the layer of material is positioned on a user of the drug delivery device.

3. The apparatus of claim 2, wherein a size of the interior cutout is larger than the drug delivery device, wherein a portion of the adhesive pad of the drug delivery device is exposed when the layer of material is placed over the drug delivery device.

4. The apparatus of claim 2, wherein the outer shape is circular.

5. The apparatus of claim 2, wherein the outer shape is elliptical.

6. The apparatus of claim 2, wherein the layer of material comprises a polyester non-woven material.

7. The apparatus of claim 2, wherein the layer of material comprises a polyethylene clear film.

8. The apparatus of claim 2, wherein a thickness of the layer of material is less than 1 millimeter (1 mm).

9. The apparatus of claim 2, wherein a strength of the adhesive positioned on the bottom surface of the layer of material is greater than a strength of an adhesive of the adhesive pad of the drug delivery device.

10. The apparatus of claim 2, wherein a strength of the adhesive positioned on the bottom surface of the layer of material is less than or equal to a strength of an adhesive of the adhesive pad of the drug delivery device.

11. The apparatus of claim 2, wherein the adhesive is an acrylic.

12. The apparatus of claim 2, wherein the drug delivery device is a wearable drug delivery device.

13. The apparatus of claim 12, wherein the wearable drug delivery device is disposable.

14. A method, comprising:
removing a release liner from a bottom surface of an adhesive overlay to expose an adhesive;
placing the adhesive overlay over a wearable drug delivery device;
attaching a first portion of adhesive overlay to an adhesive pad coupled to a bottom of the disposable drug delivery device; and
attaching a second portion of the adhesive overlap to a user of the disposable drug delivery device.

15. The method of claim 14, further comprising positioning the wearable drug delivery device through an interior cutout of the adhesive overlay.

16. The method of claim 15, wherein the adhesive overlay covers the adhesive pad.

17. The method of claim 16, wherein the adhesive overlay does not cover the wearable drug delivery device.

18. A system, comprising:
a wearable medical device;
an adhesive pad coupled to a bottom of the wearable medical device, the adhesive pad adhered to a patient; and
an adhesive overlay positioned over the wearable medical device and the adhesive pad, the wearable medical device fitting through a hole positioned in the adhesive overlay, a first portion of the adhesive overlay adhered to the adhesive pad, and a second portion of the adhesive overlay adhered to the patient.

19. The system of claim 18, wherein the hole has a shape that matches a profile of the wearable medical device.

20. The system of claim 19, wherein the shape of the hole is substantially identical to the profile of the wearable medical device.
